(19) United States
(12) Patent Application Publication
(10) Pub. No.: US 2021/0267992 A1
(43) Pub. Date: Sep. 2, 2021

(54) 5-ACETAMIDOMETHYL-OXAZOLIDINONE DERIVATIVES FOR USE IN THE TREATMENT OF CANCER

(71) Applicant: Varsity Pharmaceuticals Limited, Cambridge (GB)

(72) Inventor: James Harrison, Cambridge, Cambridgeshire (GB)

(21) Appl. No.: 17/271,864

(22) PCT Filed: Sep. 6, 2019
(86) PCT No.: PCT/GB2019/052481

§ 371 Date: Feb. 26, 2021

(30) Foreign Application Priority Data
Sep. 6, 2018 (GB) 1814487.3

(51) Int. Cl. A61K 31/5377; A61P 35/00; A61K 38/14; A61K 31/7068; A61K 31/282; A61K 33/243; A61K 31/675; A61K 31/655; A61K 31/337; A61K 31/704; A61K 31/7048; A61K 31/513; A61K 31/573; A61K 31/502; A61K 31/55; A61K 31/5025; A61K 31/454; A61K 31/80; A61K 31/7135; A61K 31/519; A61K 31/4188; A61K 31/5517

(52) U.S. Cl. CPC A61K 31/5377; A61K 31/5517; A61K 38/14; A61K 31/7068; A61K 31/282; A61K 33/243; A61K 31/675; A61K 31/655; A61K 31/337; A61K 31/704; A61K 31/7048; A61K 31/513; A61K 31/573; A61K 31/502; A61K 31/55; A61K 31/5025; A61K 31/454; A61K 31/80; A61K 31/7135; A61K 31/519; A61K 31/4188; A61P 35/00

(57) ABSTRACT

The disclosure provides a compound, or a pharmaliorating or preventing cancer.

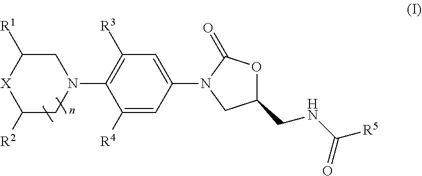

The invention relates to cancer, and in particular to novel compositions, therapies and methods for treating, preventing or ameliorating cancer.

In order to maintain genomic stability, cells have developed sophisticated signalling pathways to enable DNA damage or DNA replication stress to be resolved. Key mediators of this DNA damage response (DDR) are the serine/threonine-protein kinase ataxia telangiectasia, mutated (ATM) and ataxia telangiectasia and Rad₃-related protein (ATR) kinases, which induce cell cycle arrest and facilitate DNA repair via phosphorylation of downstream targets. Inhibiting the DDR has become an attractive therapeutic concept in cancer therapy, since (i) resistance to genotoxic therapies has been associated with increased DDR signaling, and (ii) many cancers have defects in certain components of the DDR rendering them highly dependent on the remaining DDR pathways for survival. ATM and ATR act as the apical regulators of the response to DNA double strand breaks and replication stress, respectively, with overlapping but non-redundant activities.

Highly selective small molecule inhibitors of ATM and ATR are currently in preclinical and clinical development, respectively. Preclinical data have provided a strong rationale for clinical testing of these compounds both in combination with radio- or chemotherapy, and in synthetic lethal approaches to treat tumours with deficiencies in certain DDR components. Whole genome sequencing studies have reported that mutations in DDR genes occur with a high frequency in many common tumour types, suggesting that a synthetic lethal approach with ATM or ATR inhibitors could have widespread utility. This use of ATM or ATR inhibitors could be in the form of monotherapy, or in combination with other agents targeting DDR, such as PARP inhibitors.

The present invention arises from the inventors' work in looking for new compounds (which may be ATM and ATR inhibitors) for use in treating a variety of different cancers.

In accordance with a first aspect of the invention, there is provided a compound of formula (I):

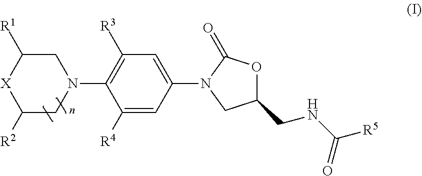

, wherein X is O, S, SO or SO₂;

R¹ is hydrogen, except when X is O then R¹ can be hydrogen, CN, CO₂R⁶ or a C₁₋₂ alkyl, optionally substituted with OR⁶, OCOR⁶, N(R⁶)₂ or NHCOR⁶;

R² is hydrogen, except when X is O and R¹ is CH₃ then R² can be H or CH₃;

R³ and R⁴ are independently hydrogen, F or Cl;

R⁵ is hydrogen, C₁₋₈ alkyl optionally substituted with one or more of R⁷; C₃₋₆ cycloalkyl, amino, C₁₋₈ alkylamino, C₁₋₈ dialkylamino or C₁₋₈ alkoxy;

each R⁶ is independently hydrogen, C₁₋₈ alkyl optionally substituted with one or more of R⁷, C₃₋₆ cycloalkyl, amino, C₁₋₈ alkylamino, C₁₋₈ dialkylamino or C₁₋₈ alkoxy;

each R⁷ is independently F, Cl, OH, C₁₋₈ alkoxy, C₁₋₈ acyloxy or O—CH₂—Ph;

and n is 0, 1 or 2;

or a pharmaceutically acceptable salt or solvate thereof, for use in treating, ameliorating or preventing cancer.

In a second aspect, there is provided a method of treating, preventing or ameliorating cancer in a subject, the method comprising administering to a subject in need of such treatment, a therapeutically effective amount of a compound of formula (I):

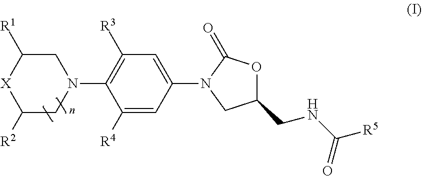

, wherein X is O, S, SO or SO₂;

R¹ is hydrogen, except when X is O then R¹ can be hydrogen, CN, CO₂R⁶ or a C₁₋₂ alkyl, optionally substituted with OR⁶, OCOR⁶, N(R⁶)₂ or NHCOR⁶;

R² is hydrogen, except when X is O and R¹ is CH₃ then R² can be H or CH₃;

R³ and R⁴ are independently hydrogen, F or Cl;

R⁵ is hydrogen, C₁₋₈ alkyl optionally substituted with one or more of R⁷; C₃₋₆ cycloalkyl, amino, C₁₋₈ alkylamino, C₁₋₈ dialkylamino or C₁₋₈ alkoxy;

each R⁶ is independently hydrogen, C₁₋₈ alkyl optionally substituted with one or more of R⁷, C₃₋₆ cycloalkyl, amino, C₁₋₈ alkylamino, C₁₋₈ dialkylamino or C₁₋₈ alkoxy;

each R⁷ is independently F, Cl, OH, C₁₋₈ alkoxy, C₁₋₈ acyloxy or O—CH₂—Ph;

and n is 0, 1 or 2;

, or a pharmaceutically acceptable salt or solvate thereof.

Advantageously, the inventors have found that compounds of formula (I) are surprisingly effective at reducing the proliferation of cancer cells.

It may be appreciated that when an element is specified in the definition of formula (I) then all isotopes of that element are also covered. For instance, the term “H” or “hydrogen” may be understood to also cover deuterium and tritium. Accordingly, in some embodiments, the compound of formula (I) may be a compound of formula (Ib):

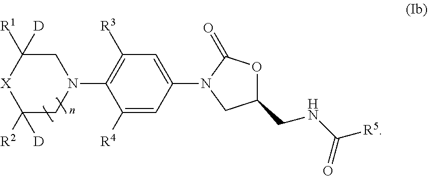

In some embodiments, R¹ and/or R² may be deuterium. In some embodiments, the compound of formula (I) may be a compound of formula (Ic):

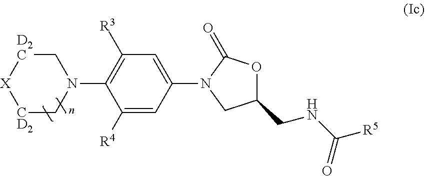

In a preferred embodiment, X is O. Accordingly, R¹ may be hydrogen, CN, CO₂R⁶ or a C₁₋₂ alkyl, optionally substituted with OR⁶, OCOR⁶, N(R⁶)₂ or NHCOR⁶. Preferably, R¹ is hydrogen, CN, CO₂H or a C₁₋₂ alkyl, optionally substituted with OH, OCOH, NH₂ or NHCOH. More preferably, R¹ is hydrogen or a C₁₋₂ alkyl. It may be appreciated that a C₁₋₂ alkyl may be methyl or ethyl. Most preferably, R¹ is hydrogen.

Preferably, R² is hydrogen.

Preferably, at least one of R³ and R⁴ is F or Cl. More preferably, one of R³ and R⁴ is F or Cl and the other is hydrogen. Most preferably, one of R³ and R⁴ is F and the other is hydrogen.

Preferably, R⁵ is hydrogen or a C₁₋₈ alkyl optionally substituted with one or more of R⁷. More preferably, R⁵ is hydrogen or a C₁₋₅ alkyl optionally substituted with one or more of R⁷. Even more preferably, R⁵ is hydrogen or a C₁₋₂ alkyl optionally substituted with one or more of R⁷. Most preferably, R⁵ is hydrogen or a C₁₋₂ alkyl. It may be appreciated that a C₁₋₂ alkyl may be methyl or ethyl. In a most preferred embodiment, R⁵ is CH₃.

Preferably, n is 1.

Accordingly, in a most preferred embodiment, the compound of formula (I) is a compound of formula (Ia):

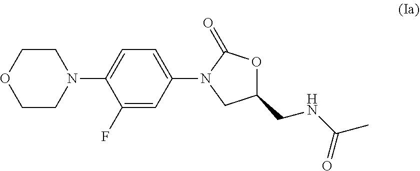 [SMILES: CC(=O)NC[C@H]1CN(c2ccc(N3CCOCC3)c(F)c2)C(=O)O1]

, or a pharmaceutically acceptable salt or solvate thereof.

It may be appreciated that the compound of formula (Ia) is linezolid.

Pharmaceutically acceptable salts include any salt of the compound of formula (I) which retains its biological properties and which is not toxic or otherwise undesirable for pharmaceutical use. The pharmaceutically acceptable salt may be derived from a variety of organic and inorganic counter-ions well known in the art.

The pharmaceutically acceptable salt may comprise an acid addition salt formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids. Alternatively, the pharmaceutically acceptable salt may comprise a base addition salt formed when an acidic proton present in the parent compound is either replaced by a metal ion, e.g., an alkali metal ion, an alkaline earth ion, an aluminium ion, alkali metal or alkaline earth metal hydroxides, such as sodium, potassium, calcium, magnesium, aluminium, lithium, zinc, and barium hydroxide, or coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

A pharmaceutically acceptable solvate refers to a compound of formula (I), or a salt thereof, that further includes a stoichiometric or non-stoichiometric amount of solvent bound by non-covalent intermolecular forces. Where the solvent is water, the solvate is a hydrate.

The cancer may be a solid tumour or solid cancer. The cancer may be bowel cancer, brain cancer, breast cancer, endometrial cancer, gastric cancer, liver cancer, lung cancer, ovarian cancer, pancreatic cancer, prostate cancer, skin cancer. The bowel cancer may be colon cancer or rectal cancer. The brain cancer may be a glioma or a glioblastoma. Any cancer from the above list may or may not carry an identified BRCA mutation. The breast cancer may be a HER2 positive breast cancer or HER2 negative breast cancer. The liver cancer may be hepatocellular carcinoma. The lung cancer may be non-small cell lung cancer or small cell lung cancer. The skin cancer may be a melanoma.

It will be appreciated that the compound of formula (I), or a pharmaceutically acceptable salt or solvate thereof, may be used in a medicament which may be used in a monotherapy (i.e. use of the compound of formula (I) alone), for treating, ameliorating, or preventing cancer. Alternatively, the compound of formula (I) or a pharmaceutically acceptable salt or solvate thereof may be used as an adjunct to, or in combination with, known therapies for treating, ameliorating, or preventing cancer.

Accordingly, the compound of formula (I) may be used in combination with a chemotherapy drug (or a combination of multiple chemotherapy drugs described herein). The chemotherapy drug may comprise bleomycin, capecitabine, carboplatin, cisplatin, cyclophosphamide, dacarbazine, docetaxel, doxorubicin, epirubicin, eribulin, etoposide, 5-fluorouracil, folinic acid, gemcitabine, methotrexate, mustine, oxaliplatin, paclitaxel, prednisolone, procarbazine, vinblastine, vincristine and/or vinorelbine. The compound of formula (I) may be for use before, after or at the same time as the chemotherapy drug. In a preferred embodiment, the compound of formula (I) is for use after the chemotherapy drug.

Alternatively, or additionally, the compound of formula (I) may be used in combination with a drug that damages DNA or which interferes with the DNA damage response process (DDR). Accordingly, the compound of formula (I) may be used in combination with a Poly (ADP-ribose) polymerase (PARP) inhibitor, an ATM inhibitor, an ATR inhibitor, a checkpoint inhibitor, a vascular endothelial growth factor (VEGF) inhibitor or a weer inhibitor. The PARP inhibitor is preferably a PARP1 inhibitor. The checkpoint inhibitor may be a programmed cell death protein 1(PD-1) inhibitor, a programmed death-ligand 1 (PD-L1) inhibitor or a cytotoxic T-lymphocyte-associated protein 4 (CTLA-4) inhibitor.

Preferably, the compound of formula (I) is used in combination with a PARP1 inhibitor. The PARP1 inhibitor may comprise a gold complex. The PARP1 inhibitor may be aurothiomalate, aurothioglucose (ATG), rucaparib, olaparib, nirparib, talazoparib, veliparib, pamiparib, 2X-121 or auranofin. The PARP1 inhibitor preferably comprises aurothiomalate, ATG or auranofin. Advantageously, the inventors have shown that the combination of a compound of formula (I) and a PARP1 inhibitor synergistically inhibits the proliferation of cancer cells. The effect is particularly noticeable when the PARP1 inhibitor is a gold complex.

Alternatively, or additionally, the compound of formula (I) may be used in combination with ionising radiation that damages DNA.

The compound of formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the compound of formula (I) described herein may be used in a number of ways. Compositions comprising the compound of formula (I) may be administered by inhalation (e.g. intranasally). Compositions may also be formulated for topical use. For instance, creams or ointments may be applied to the skin.

The compound of formula (I) according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the compound of formula (I) used according to the invention is required and which would normally require frequent administration (e.g. at least daily injection).

The compound of formula (I) and compositions according to the invention may be administered to a subject by injection into the blood stream or directly into a site requiring treatment, for example into a cancerous tumour or into the blood stream adjacent thereto. Injections may be intravenous (bolus or infusion) or subcutaneous (bolus or infusion), intradermal (bolus or infusion) or intramuscular (bolus or infusion).

In a preferred embodiment, the compound of formula (I) is administered orally. Accordingly, the compound of formula (I) may be contained within a composition that may, for example, be ingested orally in the form of a tablet, capsule or liquid.

It will be appreciated that the amount of the compound of formula (I) that is required is determined by its biological activity and bioavailability, which in turn depends on the mode of administration, the physiochemical properties of the compound of formula (I), and whether it is being used as a monotherapy, or in a combined therapy. The frequency of administration will also be influenced by the half-life of the compound of formula (I) within the subject being treated. Optimal dosages to be administered may be determined by those skilled in the art, and will vary with the particular inhibitor in use, the strength of the pharmaceutical composition, the mode of administration, and the advancement of the cancer. Additional factors depending on the particular subject being treated will result in a need to adjust dosages, including subject age, weight, gender, diet, and time of administration.

The compound of formula (I) may be administered before, during or after onset of the cancer to be treated. Daily doses may be given as a single administration. However, preferably, the compound of formula (I) is given two or more times during a day, and most preferably twice a day.

Generally, a daily dose of between 0.01 μg/kg of body weight and 500 mg/kg of body weight of the compound of formula (I) according to the invention may be used for treating, ameliorating, or preventing cancer. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

A patient receiving treatment may take a first dose upon waking and then a second dose in the evening (if on a two dose regime) or at 3- or 4-hourly intervals thereafter. Alternatively, a slow release device may be used to provide optimal doses of the compound of formula (I) to a patient without the need to administer repeated doses.

Known procedures, such as those conventionally employed by the pharmaceutical industry (e.g. in vivo experimentation, clinical trials, etc.), may be used to form specific formulations comprising the compound of formula (I) according to the invention and precise therapeutic regimes (such as daily doses of the compound of formula (I) and the frequency of administration). The inventors believe that they are the first to describe a pharmaceutical composition for treating cancer, based on the compound of formula (I).

Hence, in a third aspect of the invention, there is provided a pharmaceutical composition for treating cancer comprising a compound of formula (I), as defined in the first aspect, or a pharmaceutically acceptable salt or solvate thereof, and a pharmaceutically acceptable vehicle.

The pharmaceutical composition can be used in the therapeutic amelioration, prevention or treatment in a subject of cancer.

The compounds of Formula (I) is as defined in relation to the first and second aspects. Preferably, the compound of formula (I) is a compound of formula (Ia), as defined herein, or a pharmaceutically acceptable salt or solvate thereof. It may be appreciated that the compound of formula (Ia) is linezolid.

The pharmaceutical composition may further comprise a drug that damages DNA or which interferes with the DNA damage response process (DDR). The DDR drug may be a Poly (ADP-ribose) polymerase (PARP) inhibitor, an ATM inhibitor, an ATR inhibitor, a checkpoint inhibitor, a vascular endothelial growth factor (VEGF) inhibitor or a weer inhibitor. The PARP inhibitor is preferably a PARP1 inhibitor. The checkpoint inhibitor may be a programmed cell death protein 1(PD-1) inhibitor, a programmed death-ligand 1 (PD-L1) inhibitor or a cytotoxic T-lymphocyte-associated protein 4 (CTLA-4) inhibitor.

The PARP inhibitor may be as defined in relation to the first and second aspects. Preferably, the PARP inhibitor is a PARP1 inhibitor. The PARP1 inhibitor may comprise a gold complex. The PARP1 inhibitor may be aurothiomalate, aurothioglucose (ATG), auranofin, rucaparib, olaparib, nirparib, talazoparib, veliparib, pamiparib or 2X-121. The PARP1 inhibitor preferably comprises aurothiomalate, ATG or auranofin.

The invention also provides, in a fourth aspect, a process for making the composition according to the third aspect, the process comprising contacting a therapeutically effective amount of compound of formula (I), as defined in the first aspect, or a pharmaceutically acceptable salt or solvate thereof, and a pharmaceutically acceptable vehicle.

A “subject” may be a vertebrate or mammal. Most preferably, the subject is a human being.

A “therapeutically effective amount” of the compound of formula (I) is any amount which, when administered to a subject, is the amount of drug that is needed to treat the cancer.

For example, the therapeutically effective amount of the compound of formula (I) used may be from about 0.01 mg to about 800 mg, and preferably from about 0.01 mg to about 500 mg. It is preferred that the amount of the compound of formula (I) is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the compound of formula (I)) according to the invention. In tablets, the compound of formula (I) may be mixed with a vehicle having the necessary compression properties in suitable proportions and compacted in the shape and size desired. The powders and tablets preferably contain up to 99% of the compound of formula (I). Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compound of formula (I) according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound of formula (I) may be prepared as a sterile solid composition that may be dissolved or suspended at the time of administration using sterile water, saline, or other appropriate sterile injectable medium.

The compound of formula (I) and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compound of formula (I) used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

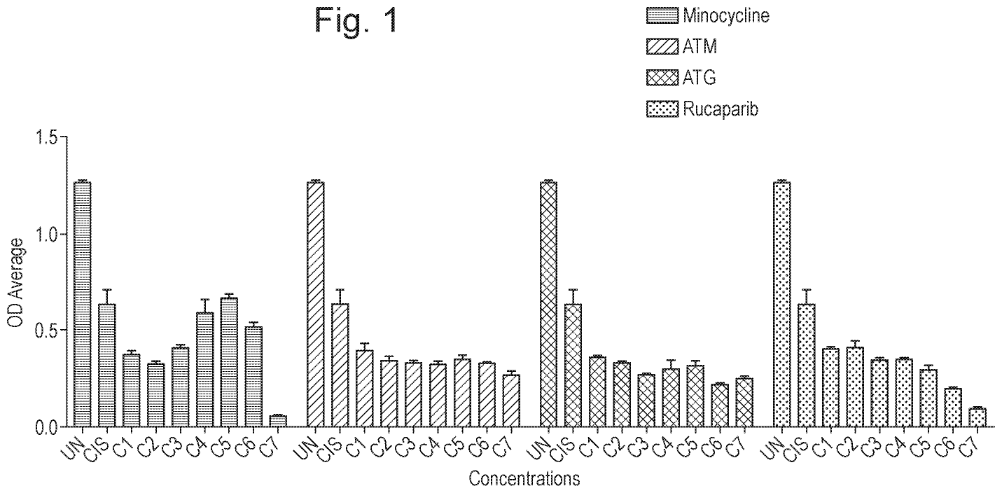

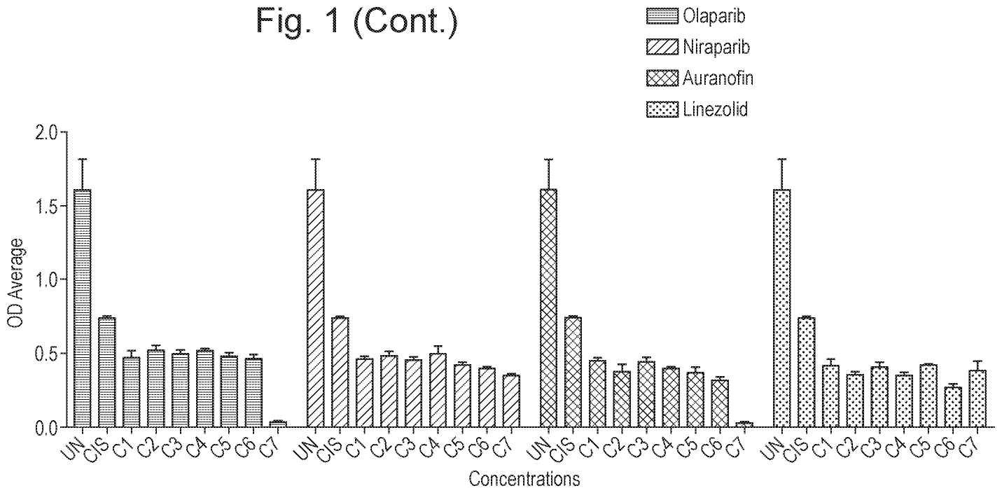

FIG. 1 is a graph showing the absorbance values BRCA1 deficient ovarian cancer cells, UWB1.289, exposed to cisplatin 1 μM for 24 hours followed by Minocycline, Aurothiomalate (ATM), Aurothioglucose (ATG), Rucaparib, Olaparib, Nirparib, Auranofin or Linezolid for 6 days;

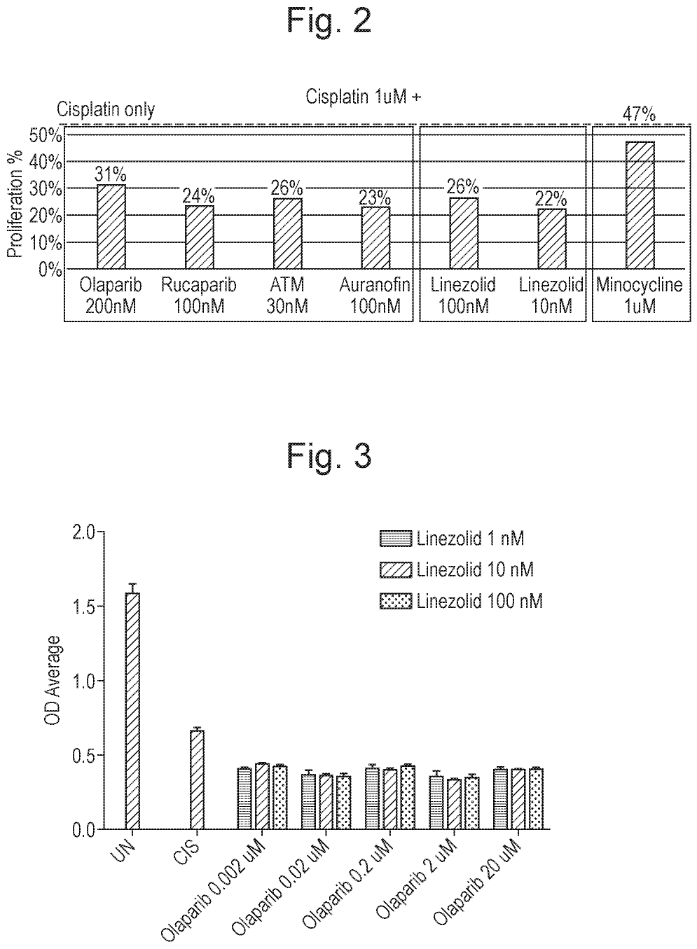

FIG. 2 is a graph showing the percentage proliferation of the UWB1.289 cell line shown in FIG. 1 for selected experiments;

FIG. 3 is a graph showing the absorbance values of the UWB1.289 cell line after it was exposed to cisplatin 1 μM for 24 hours and followed by a combination of Olaparib and Linezolid for 6 days;

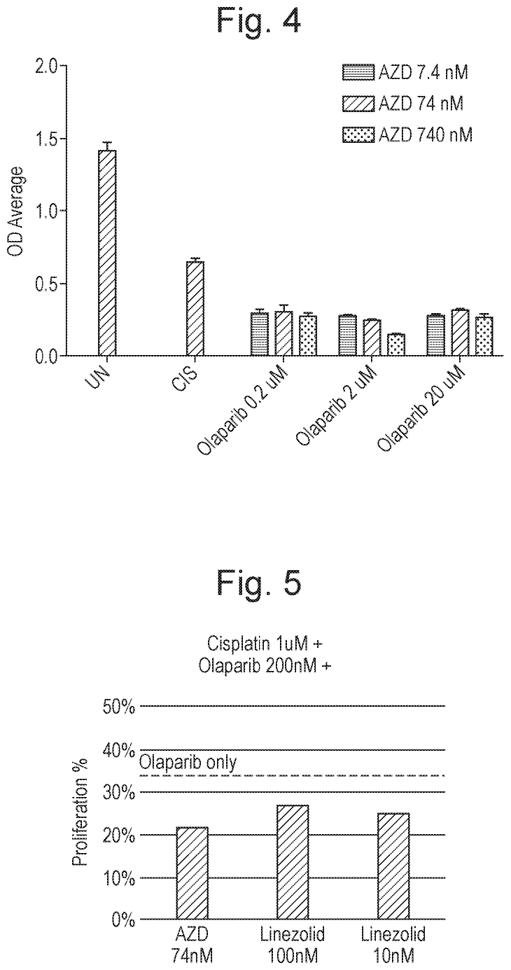

FIG. 4 is a graph showing the absorbance values of the UWB1.289 cell line after it was exposed to cisplatin 1 μM for 24 hours and followed by a combination of Olaparib and AZD6738 (AZD) for 6 days;

FIG. 5 is a graph showing the percentage proliferation of the UWB1.289 cell line shown in FIGS. 3 and 4 for selected experiments;

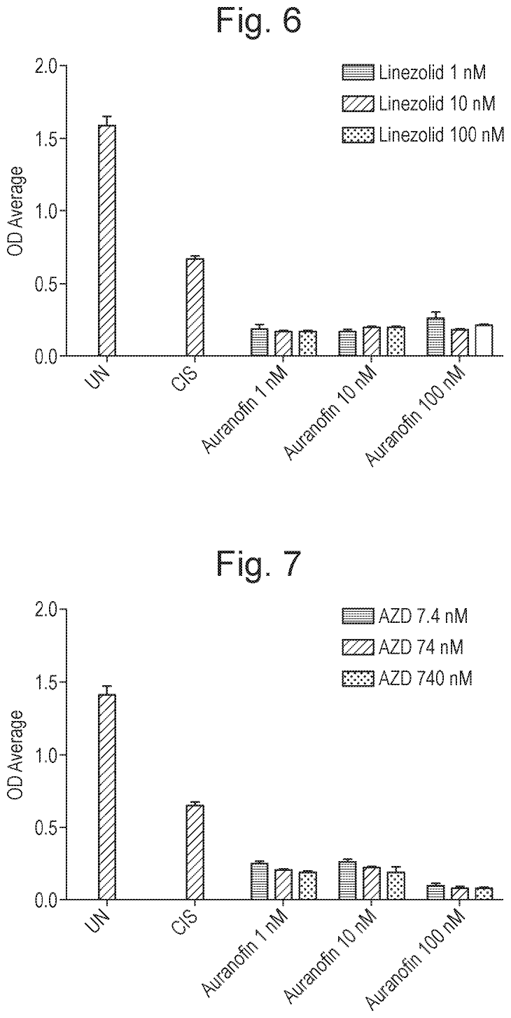

FIG. 6 is a graph showing the absorbance values of the UWB1.289 cell line after it was exposed to cisplatin 1 μM for 24 hours and followed by a combination of Auranofin and Linezolid for 6 days;

FIG. 7 is a graph showing the absorbance values of the UWB1.289 cell line after it was exposed to cisplatin 1 μM for 24 hours and followed by a combination of Auranofin and AZD6738 (AZD) for 6 days;

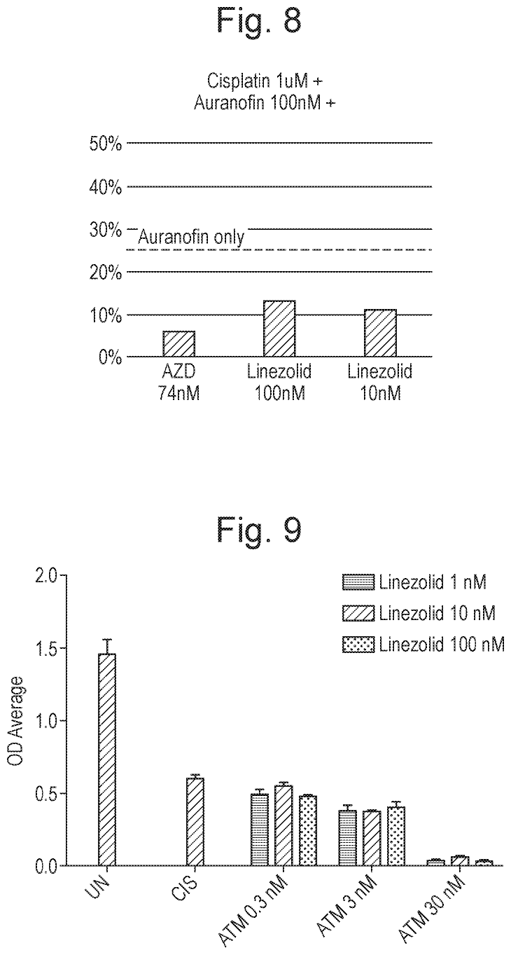

FIG. 8 is a graph showing the percentage proliferation of the UWB1.289 cell line shown in FIGS. 6 and 7 for selected experiments;

FIG. 9 is a graph showing the absorbance values of the UWB1.289 cell line after it was exposed to cisplatin 1 μM for 24 hours and followed by a combination of Aurothiomalate (ATM) and Linezolid for 6 days;

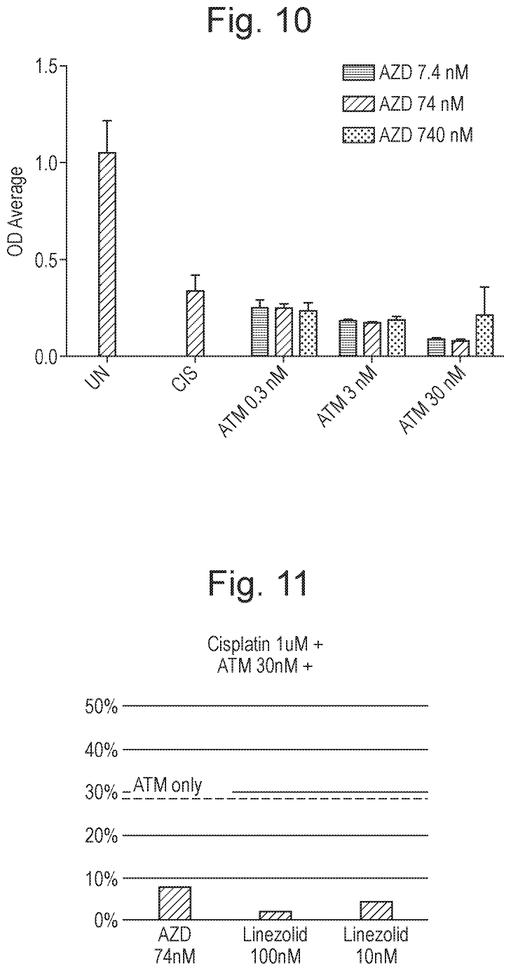

FIG. 10 is a graph showing the absorbance values of the UWB1.289 cell line after it was exposed to cisplatin 1 μM for 24 hours and followed by a combination of Aurothiomalate (ATM) and AZD6738 (AZD) for 6 days;

FIG. 11 is a graph showing the percentage proliferation of the UWB1.289 cell line shown in FIGS. 9 and 10 for selected experiments; and

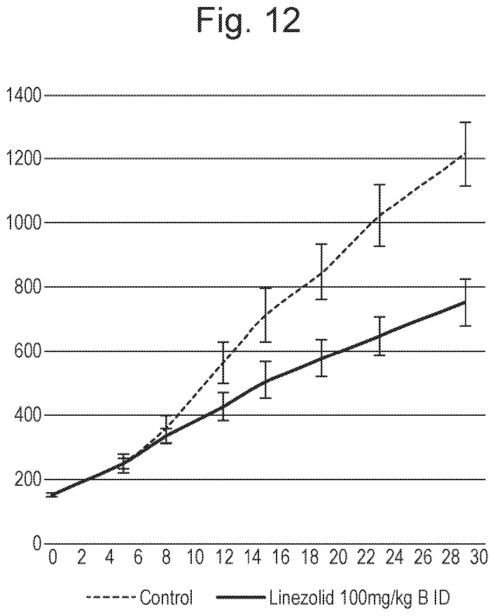

FIG. 12 is a graph showing the size of tumours in mice in a MDA-MB-436 (BRCA1 mutation) breast cancer cell xenograft experiment, where the mice are either untreated or are treated with 100 mg/kg of linezolid BID.

### EXAMPLE 1

### Comparison of Ability of PARPis and ATR Inhibitors to Reduce Proliferation of Cancer Cells

Methods
Cell morphology, viability and proliferation rate was assessed by visual and counting method.
Day 0: Cells were split and around 1000 cells/well were seeded in normal complete media.
Day 1: Cisplatin was added at 1 μM to the test and controls wells. Untreated cells were left as control.
Day 2: medium containing cisplatin was discarded and new fresh media containing bromodeoxyuridine (BrdU) and the drugs at the concentrations shown in table 1, below, was added.
Day 6: medium was discarded, and cells fixed. BrdU assay was performed according to the manufacturer.
Controls comprised cells without BrdU (Blank), untreated cells (UN) and Cisplatin treated cells for 24 hours only (CIS).
All experiments were conducted in triplicate.

TABLE 1
Concentrations of drugs in medium added to cells

| | Conc | Conc | Conc | Conc | Conc | Conc | Conc |
|---|---|---|---|---|---|---|---|
| Compound | 1 | 2 | 3 | 4 | 5 | 6 | 7 |
| Minocycline | 0.5 nM | 5 nM | 50 nM | 500 | 5 | 50 μM | 500 |
| | | | | nM | μM | | μM |
| Aurothiomalate | 0.05 | 0.5 nM | 5 nM | 50 | 500 | 5 μM | 50 |
| (ATM) | nM | | | nM | nM | | μM |
| ATG | 0.0012 | 0.012 | 0.12 | 1.2 | 12 | 120 | 1.2 |
| | nM | nM | nM | nM | nM | nM | μM |
| Rucaparib | 0.01 | 0.1 nM | 1 nM | 10 | 100 | 1 μM | 10 |
| | nM | | | nM | nM | | μM |
| Olaparib | 1 nM | 10 nM | 100 | 1 | 10 | 100 | 1 |
| | | | nM | μM | μM | μM | mM |
| Niraparib | 0.00002 | 0.0002 | 0.002 | 0.02 | 0.2 | 2 nM | 20 |
| | nM | nM | nM | nM | nM | | nM |
| Auranofin | 0.01 | 0.1 nM | 1 nM | 10 | 100 | 1 μM | 10 |
| | nM | | | nM | nM | | μM |
| Linezolid | 0.01 | 0.1 nM | 1 nM | 10 | 100 | 1 μM | 10 |
| | nM | | | nM | nM | | μM |

BrdU Proliferation Assay
After 6 days of incubation with drugs, the media was discarded, and cells were fixed for 30 minutes, at room temperature, with FixDenat Solution.
FixDenat Solution was then removed and substituted with Anti-BrdU-POD working solution for 2 hours at room temperature.
Plates were then washed 3 times with Washing buffer.
Substrate solution was added.
The reaction was stopped by adding H2SO4 and immediately read at 450 nm.

Data Analysis

Data was analysed using Excel and Prism software. The average of the absorbances and the standard error were calculated using the technical triplicate for each condition.

Results

Olaparib, rucaparib and niraparib are all known and approved PARPis. Unsurprisingly, the results show that these compounds were able to effectively reduce proliferation of the cancer cells. However, these approved PARPis do not reduce proliferation close to zero.

ATM, ATG and auranofin are all gold complexes that can act as PARPis. FIGS. 1 and 2 show that these compounds were also able to effectively reduce proliferation of the cancer cells. The proliferation reduction caused by the gold complexes is approximately equivalent to the proliferation reduction achieved by the approved PARPis.

Meanwhile, minocycline is a selective PARP2 inhibitor. As shown in FIGS. 1 and 2, this compound was not able to effectively reduce proliferation of the cancer cells, except at high concentrations. This is consistent with the observation that PARP1 is required for DDR.

Finally, the results show that linezolid achieves a similar decrease in proliferation of cancer cells to the approved PARPis and the gold complexes.

### EXAMPLE 2

### Combining PARPis and ATR Inhibitors to Reduce Proliferation of Cancer Cells

Methods

The methods were the same as described in example 1 except that the new fresh media added on day 2 contained bromodeoxyuridine (BrdU) and the drugs at the concentrations shown in table 2, below. All possible combinations of concentrations for compounds A and B were tested.

TABLE 2
Concentrations of drugs in medium added to cells

| Compound | Concentration | Compound | Concentration |
|---|---|---|---|
| A | of compound A | B | of compound B |
| Olaparib | 2 nM, 20 nM, | Linezolid | 1 nM, 10 nM and |
| | 200 nM, 2 μM and | | 100 nM |
| | 20 μM | | |
| Olaparib | 200 nM, 2 μM | AZD | 7.4 nM, 74 nM |
| | and 20 μM | | and 740 nM |
| Aurothiomalate | 0.3 nM, 3 nM and | Linezolid | 1 nM, 10 nM and |
| (ATM) | 30 nM | | 100 nM |
| Aurothiomalate | 0.3 nM, 3 nM and | AZD | 7.4 nM, 74 nM |
| (ATM) | 30 nM | | and 740 nM |
| Auranofin | 1 nM, 10 nM and | Linezolid | 1 nM, 10 nM and |
| | 100 nM | | 100 nM |
| Auranofin | 1 nM, 10 nM and | AZD | 7.4 nM, 74 nM |
| | 100 nM | | and 740 nM |

Results

The results are shown in FIGS. 3 to 11. Due to the fact that approved PARPis, gold complexes and ATR inhibitors do not reduce proliferation close to zero, the inventors have considered combination treatments in order to evaluate the possibility of additional, synergistic proliferation reduction of combination regimes in comparison to individual drug therapy.

AZD6738 (AZD) is a known ATR inhibitor. As expected, the combination of olaparib and AZD resulted in a further decreased proliferation when compared to the degree of proliferation observed for olaparib alone, see FIG. 5. The addition of linezolid, instead of AZD, also showed a similar decrease in proliferation.

The combination of auranofin or Aurothiomalate (ATM) with linezolid or AZD showed a particularly marked improvement over the gold complexes when used alone, see FIG. 11. In fact, proliferation was reduced to 2% when Aurothiomalate (ATM) was present at a concentration of 30 nM and linezolid was present at a concentration of 100 nM.

### EXAMPLE 3

### MDA-MB-436 (BRCA1 Mutation) Breast Cancer Cell Xenograft Experiment

Animal Maintenance

Animals were quarantined for 7 days before the study. The general health of the animals was evaluated by a veterinarian, and complete health checks were performed. Animals with abnormalities were excluded prior the study.

Housing

General procedures for animal care and housing were in accordance with the standard, Commission on Life Sciences, National Research Council, Standard operating procedures (SOPs) of Pharmaron, Inc. The mice were kept in laminar flow rooms at constant temperature and humidity with 3-5 mice in each cage. Animals were housed in polycarbonate cage which is in the size of 300×180×150 mm³ and in an environmentally monitored, well-ventilated room maintained at a temperature of (22±3° C.) and a relative humidity of 40% to 70%. Fluorescent lighting provided illumination approximately 12 hours per day. The bedding material was soft wood, which was changed once per week.

Diet

Animals had free access to irradiation sterilized dry granule food during the entire study period except for time periods specified by the protocol.

Water

Sterile drinking water in a bottle was available to all animals ad libitum during the quarantine and study periods. The bottle and the stopper with attached sipper tube was autoclaved prior to use. Samples of water from the animal facility were analyzed and results of water analysis were reviewed by the veterinarian, or designee, to assure that no known contaminants were present that could have interfered with or affected the outcome of studies.

Method for Tumour Inoculation

The MDA-MB-436 tumour cell line was maintained in vitro as monolayer culture in DMEM medium modified supplemented with 10% heat inactivated foetal bovine serum at 37° C. in an atmosphere of 5% CO₂ in air. The tumour cells were routinely sub-cultured once a week by trypsin-EDTA treatment, not to exceed 4-5 passages. The cells growing in an exponential growth phase were harvested and counted for tumour inoculation.

All mice were inoculated subcutaneously on the right flank with MDA-MB-436 tumour cells (1×10⁷) in 0.1 ml of DMEM with Matrigel mixture (1:1 ratio) for tumour development. The treatment started when the mean tumour size reached is approximately 100-150 mm³. Mice were then be assigned to groups such that the mean tumour volume is the same for each treatment group. The treatments were administered to the tumour-bearing mice orally at 12 hour intervals.

Formulation

560 mg linezolid was dissolved in 1.4 ml ethanol and 12.6 ml PEG400. The solution was vortexed and sonicated with high energy ultrasonic probe to get a uniform solution. The resultant solution was used for 1 day.

Tumour Measurements

The measurement of tumour size was conducted twice weekly with a calliper and recorded. The tumour volume (mm3) was estimated using the formula: TV=a×b²/2, where “a” and “b” are long and short diameters of a tumour, respectively.

Results

As shown in FIG. 12, the mice treated with 100 mg/kg of linezolid BID had a reduced tumour size of 38% compared to the control group.

It is noted that the dose 100 mg/kg BID of linezolid administered to the mice is equivalent to a human dose. This is generally a dosage of 600 mg BID, either orally or by i.v., in humans.

Conclusions

Linezolid has been shown to reduce proliferation of cancer cells when used alone, for both in vitro and in vivo experiments. Furthermore, a synergistic effect is observed when linezolid is used with a PARPi. A particularly noticeable synergistic effect was observed for the combination of ATM and linezolid. The inventors conclude that proliferation reduction due to linezolid is equivalent to proliferation reduction achieved by AZD6738, arguably the ATR inhibitor that is currently the most advanced in its clinical trial programme.

1. A method of treating, preventing or ameliorating cancer in a subject in need thereof, the method comprising administering to the subject a therapeutically effective amount of a compound of formula (I): 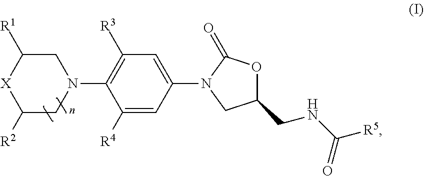
wherein X is O, S, SO or SO₂;
R¹ is hydrogen, except when X is O then R¹ can be hydrogen, CN, CO₂R⁶ or a C₁₋₂ alkyl, optionally substituted with OR⁶, OCOR⁶, N(R⁶)₂ or NHCOR⁶;
R² is hydrogen, except when X is O and R¹ is CH₃ then R² can be H or CH₃;
R³ and R⁴ are independently hydrogen, F or Cl;
R⁵ is hydrogen, C₁₋₈ alkyl optionally substituted with one or more of R⁷; C₃₋₆ cycloalkyl, amino, C₁₋₈ alkylamino, C₁₋₈ dialkylamino or C₁₋₈ alkoxy;
each R⁶ is independently hydrogen, C₁₋₈ alkyl optionally substituted with one or more of R⁷, C₃₋₆ cycloalkyl, amino, C₁₋₈ alkylamino, C₁₋₈ dialkylamino or C₁₋₈ alkoxy;
each R⁷ is independently F, Cl, OH, C₁₋₈ alkoxy, C₁₋₈ acyloxy or O—CH₂—Ph;
and n is 0, 1 or 2;
or a pharmaceutically acceptable salt or solvate thereof.

2. The method of claim 1, wherein X is O.

3. The method of claim 1, wherein R¹ is hydrogen, CN, CO₂R⁶ or a C₁₋₂ alkyl, optionally substituted with OR⁶, OCOR⁶, N(R⁶)₂ or NHCOR⁶, optionally wherein R¹ is hydrogen, CN, CO₂H or a C₁₋₂ alkyl, optionally substituted with OH, OCOH, NH₂ or NHCOH.

4. (canceled)

5. The method of claim 1, wherein R¹ is hydrogen or a C₁₋₂ alkyl.

6. The method of claim 1, wherein R² is hydrogen.

7. The method of claim 1, wherein at least one of R³ and R⁴ is F or Cl.

8. The method of claim 1, wherein one of R³ and R⁴ is F or Cl and the other is hydrogen, optionally one of R³ and R⁴ is F and the other is hydrogen.

9. The method of claim 1, wherein R⁵ is hydrogen or a C₁₋₈ alkyl optionally substituted with one or more of R⁷.

10. The method of claim 1, wherein R⁵ is hydrogen or a C₁₋₅ alkyl optionally substituted with one or more of R⁷, optionally wherein R⁵ is CH₃.

11. (canceled)

12. The method of claim 1, wherein n is 1.

13. The method of claim 1, wherein the compound of formula (I) is a compound of formula (Ia): 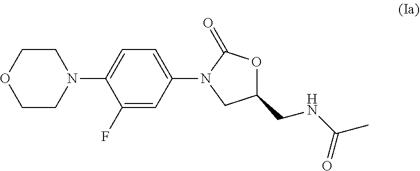 [SMILES: CC(=O)NC[C@H]1CN(c2ccc(N3CCOCC3)c(F)c2)C(=O)O1]
or a pharmaceutically acceptable salt or solvate thereof.

14. The method of claim 1, wherein the cancer is a solid tumour or solid cancer.

15. The method of claim 1, wherein the cancer is bowel cancer, brain cancer, breast cancer, endometrial cancer, gastric cancer, liver cancer, lung cancer, ovarian cancer, pancreatic cancer, prostate cancer or skin cancer, optionally wherein: (i) the bowel cancer is colon cancer or rectal cancer; (ii) the brain cancer is a glioma or a glioblastoma; (iii) the breast cancer is a HER2 positive breast cancer or HER2 negative breast cancer; (iv) the liver cancer is hepatocellular carcinoma; (v) the lung cancer is non-small cell lung cancer or small cell lung cancer; or (vi) the skin cancer is a melanoma.

16. (canceled)

17. The method of claim 1, wherein the compound of formula (I) is used in combination with one or more chemotherapy drugs, optionally wherein the compound of formula (I) is administered after the one or more chemotherapy drugs.

18. The method of claim 17, wherein the chemotherapy drug comprises bleomycin, capecitabine, carboplatin, cisplatin, cyclophosphamide, dacarbazine, docetaxel, doxorubicin, epirubicin, eribulin, etoposide, 5-fluorouracil, folinic acid, gemcitabine, methotrexate, mustine, oxaliplatin, paclitaxel, prednisolone, procarbazine, vinblastine, vincristine and/or vinorelbine.

19. The method of claim 1, wherein the compound of formula (I) is used in combination with a drug that damages DNA or which interferes with the DNA damage response process (DDR).

20. The method of claim 19, wherein the compound of formula (I) is used in combination with a Poly (ADP-ribose) polymerase (PARP) inhibitor, an ATM inhibitor, an ATR inhibitor, a checkpoint inhibitor, a vascular endothelial growth factor (VEGF) inhibitor or a wee1 inhibitor.

21. The method of claim 20, wherein (i) the PARP inhibitor is a PARP1 inhibitor; or (ii) the checkpoint inhibitor is a programmed cell death protein 1 (PD-1) inhibitor, a programmed death-ligand 1 (PD-L1) inhibitor or a cytotoxic T-lymphocyte-associated protein 4 (CTLA-4) inhibitor.

22. The method of claim 21, wherein the PARP1 inhibitor is aurothiomalate, aurothioglucose (ATG), rucaparib, olaparib, nirparib, talazoparib, veliparib, pamiparib, 2X-121 or auranofin.

23. The method of claim 22, wherein the PARP1 inhibitor comprises a gold complex, optionally wherein the PARP1 inhibitor comprises aurothiomalate, ATG or auranofin.

24.-29. (canceled)
